(19) United States
(12) Patent Application Publication
(10) Pub. No.: US 2003/0114832 A1
(43) Pub. Date: Jun. 19, 2003

(54) Interventional catheter with three dimensional articulation

(76) Inventors: Robert Edward Kohler, Lake Elmo, MN (US); Michael Gregory O'Connor, Lino Lakes, MN (US); Sean Patrick Skubitz, Shoreview, MN (US)

Correspondence Address: MERCHANT & GOULD PC, P.O. BOX 2903, MINNEAPOLIS, MN, 55402-0903, US

(21) Appl. No.: 10/023,314

(22) Filed: Dec. 14, 2001

(51) Int. Cl. A61M 25/01

(52) U.S. Cl. 604/528

(57) ABSTRACT

The present invention relates to a catheter incorporating an articulating distal end, including a catheter body with a central lumen and an outer wall in which are one or more wires, and a handle including mechanisms for applying tension to the wires. Applying tension to a wire will cause a bending portion of the catheter to bend into an arc. When more than one wire or tensioning member and more than one mechanism for applying tension are included in the catheter, the wires are offset from each other so that multiple bends on multiple planes may be formed in multiple bending portions in the body of the catheter. The present invention further relates to a catheter including one wire, and a handle including a mechanism for applying tension to the wire, the wire and the catheter body configured so that applying tension to a wire will cause more than one bending portion of the catheter to bend into an arc, and the bending portions are made of different durometer material. The present invention further relates to a method of using a catheter to access a heart chamber of a patient and locate the distal end of the catheter proximate a shunt located in a wall of the heart.

### FIELD OF THE INVENTION

The present invention relates to a catheter with a tip that can be articulated within the body of a patient.

### BACKGROUND OF THE INVENTION

It is known to place shunts or stents within a heart wall of a patient to permit oxygenated blood to flow directly from a heart chamber into a coronary blood vessel at a point downflow of an occlusion within the vessel. Such devices and methods for placing them are detailed in U.S. Pat. Nos. 5,755,682, 5,908,029 and 5,944,019. Once these devices have been placed within the heart wall, diagnostic procedures involving the devices are sometimes necessary. To perform these procedures, a device that allows stabilization within the heart chamber and alignment with the device is useful. Improvements to existing catheters to provide the ability to stabilize the catheter within the heart chamber and facilitate the alignment of the catheter with the device in the heart wall are desirable.

### SUMMARY OF THE INVENTION

One aspect of the present invention relates to a catheter incorporating an articulating distal end. The catheter includes a catheter body with a central lumen and an outer wall in which are one or more wires or other tensioning members, and a handle including mechanisms for applying tension to the wires, the wires and the catheter body configured so that applying tension to a wire will cause a bending portion of the catheter to bend into an arc. When more than one wire or tensioning member and more than one mechanism for applying tension are included in the catheter, the wires are offset from each other so that multiple bends on multiple planes may be formed in multiple bending portions in the body of the catheter. Another aspect of the present invention relates to a catheter including a catheter body with a central lumen and an outer wall in which are one wire or other tensioning member, and a handle including a mechanism for applying tension to the wire, the wire and the catheter body configured so that applying tension to a wire will cause more than one bending portion of the catheter to bend into an arc, the bending portions including material of different durometer. A further aspect of the present invention relates to a method of using a catheter with a central lumen to access a heart chamber of a patient and locate the distal end of the catheter proximate a shunt located in a wall of the heart. The catheter is inserted into the heart chamber and tension is applied to one or more wires or tensioning members within an outer wall of the catheter to bend bending portions of the catheter and permit a distal end of the catheter to positioned proximate the shunt.

### BRIEF DESCRIPTION OF THE DRAWINGS

The accompanying drawings, which are incorporated in and constitute a part of the description, illustrate several aspects of the invention and together with the description, serve to explain the principles of the invention. A brief description of the drawings is as follows:

FIG. 1 is a side view of an embodiment of a catheter in accordance with the present invention.

FIG. 2 is a cross-sectional view of the catheter of FIG. 1 taken at line 2-2.

FIG. 3 is a cross-sectional view of an alternative embodiment of a catheter in accordance with the present invention including four deflection wires.

FIG. 4 is a cross-sectional view of an alternative embodiment of the catheter of FIG. 2 in accordance with the present invention including two flat deflection tensioning members.

FIG. 5 is a cross-sectional view of an alternative embodiment of the catheter of FIG. 3 in accordance with the present invention including four flat deflection tensioning members.

FIG. 6 is a closer detail view of the distal end of the catheter of FIG. 1.

FIG. 7 is a cross-sectional view of the catheter of FIG. 3, taken along line 7-7.

FIG. 8 is a closer detail view of the distal end of the catheter of FIG. 1, showing both bending portions bent.

FIG. 9 is a view of the catheter of FIG. 8 with the first bend portion bent at a more acute angle.

FIG. 10 is a view of the catheter of FIG. 9 with the first bend portion bent at a more acute angle.

FIG. 11 is a schematic illustration with a heart in partial cutaway of the catheter of FIG. 2 used to catheterize the left ventricle of a patient's heart via the femoral artery.

FIG. 12 is a close-up of the heart of FIG. 11, showing a distal end of the catheter within the patient's left ventricle and a shunt in place in the wall of the patient's heart.

FIG. 13 is a schematic representation of the top cross-sectional view of the patient's left ventricle of FIG. 12, with the distal end of the catheter extending into the heart.

FIG. 14 is the heart and catheter of FIG. 13 with the first bending portion of the catheter bent in an arc.

FIG. 15 is the heart and catheter of FIG. 14 with the second bending portion of the catheter bent into an arc but not yet aligned with the shunt.

FIG. 16 is the heart and catheter of FIG. 15 with the distal end aligned with the shunt and a collapsible basket extending from with the catheter projecting over a leg of the shunt protruding into the left ventricle.

FIG. 17 is a side view of a handle in accordance with the present invention for use with the catheter of FIG. 1, with provision to apply tension to a single deflection wire.

FIG. 18 is a cross-sectional view of the handle of FIG. 17.

FIG. 19 is a cross-sectional view of an alternative handle for use with the catheter of FIG. 1 with provision to apply tension to two deflection wires.

FIG. 20 is a cross-sectional view of another alternative handle for use with the catheter of FIG. 1 with provision to apply tension to a single deflection wire by a ratcheting mechanism.

FIG. 21 is a cross-sectional view of a third alternative handle for use with the catheter of FIG. 1 with provision to apply tension to a single deflection wire by an alternative ratcheting mechanism.

FIG. 22 is a cross-sectional view of a fourth alternative handle for use with the catheter of FIG. 1 with provision to apply tension to two deflection wires with the ratcheting mechanism of FIG. 20.

FIG. 23 is a cross-sectional view of a fifth alternative handle for use with the catheter of FIG. 1 with provision to apply tension to two deflection wires with the ratcheting mechanism of FIG. 21.

### DETAILED DESCRIPTION

With reference to the detailed drawing figures in which identical elements are numbered identically throughout, a description of the preferred embodiment and various alternative embodiments will now be provided.

Once a shunt has been placed in the heart wall as described in U.S. Pat. No. 5,944,019, there may arise the need to access the shunt for diagnostic or other reasons. For example, a physician may desire to inject radio-opaque chemical contrast material through the shunt to permit the use of cardiac imaging techniques to verify blood flow through the affected coronary artery downstream of the site of the shunt. Alternatively, it may be desirable to reach through the shunt to insert angioplasty tools to a site in the affected coronary artery downstream of the site of the shunt. Further, a physician may access the shunt to insert an arterial stent into the affected coronary artery at a site downstream from the shunt.

One of the least traumatic methods of accessing the heart and any shunts that might be implanted in the heart wall is with a catheter which enters the body via insertion through the femoral artery in the patient's groin and is advanced through the femoral artery, descending aorta and ascending aorta, into the heart. Catheters for femoral insertion are known. However, when accessing a shunt placed in the heart wall of a patient without cardiopulmonary bypass, actually inserting a tool or other device into the shunt and the artery downstream of the shunt can be quite difficult. Without cardiopulmonary bypass, the patient's heart must necessarily be contracting during the catheterization, making the environment around the shunt quite dynamic. Known catheterization methods and apparatus do not address this issue.

The present invention relates to a technique and devices for accessing shunts through heart walls. One aspect of the present invention relates to a technique and apparatus for allowing a catheter to enter the heart and align with or attach to an object imbedded in the heart wall with a high degree of certainty while a normal cardiac rhythm is maintained.

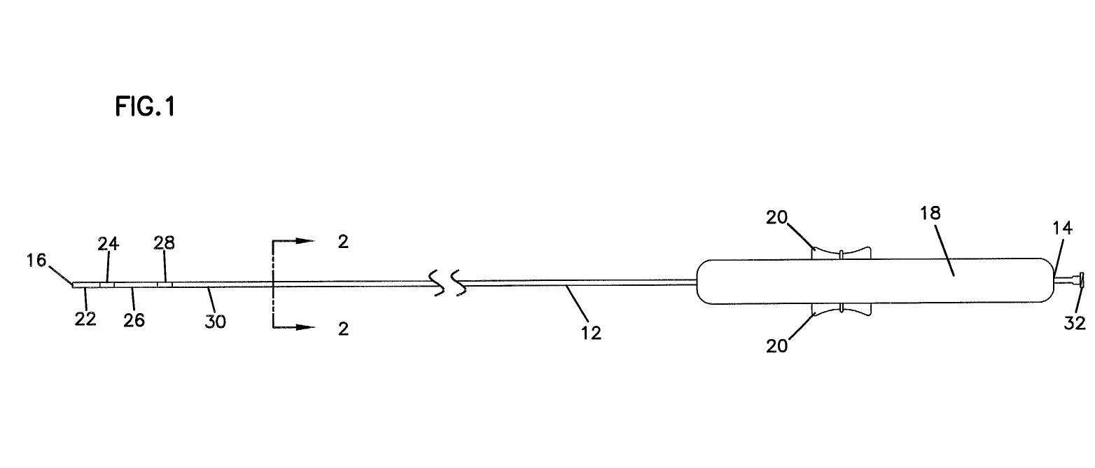

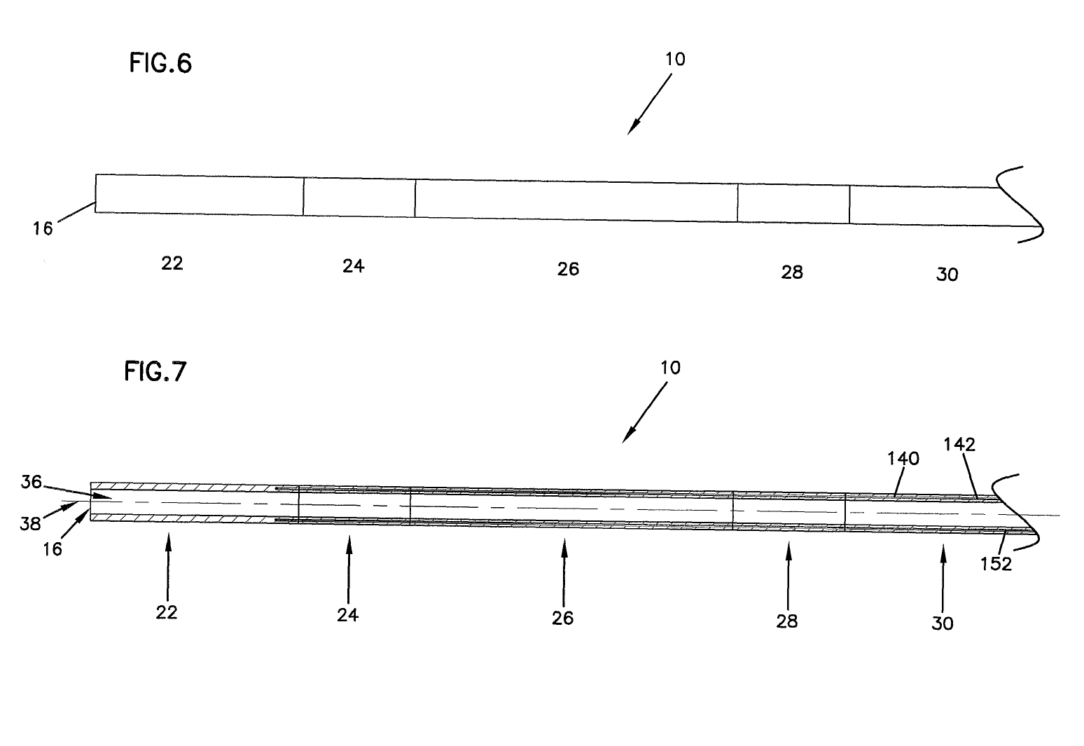

Referring now to FIGS. 1 and 6, a catheter 10 with a catheter body 12 including a proximal end 14 and a distal 16 is shown. Attached to catheter 10 at proximal end 14 is a handle 18. Catheter body 12 includes a distal segment 22, a first bending portion 24, a mid segment 26, a second bending portion 28 and a proximal segment 30. Segments 22, 26 and 30 are made from a flexible material to permit catheter 10 to be introduced into a patient's vascularity and maneuvered to a desired location without damaging the vessels. Bending portions 24 and 28 are made of a similar material with a lower durometer than segments 22, 26 and 30, meaning pending portions 24 and 28 are more easily bent, while segments 22, 26 and 30 are relatively more rigid. Bending portions 24 and 28 may be made of material of the same durometer, alternatively, first bending portion 24 may be made of a lower durometer material than second bending portion 28. Handle 18 includes two tensioning mechanisms 20, discussed in more detail below, and an access port 32 allowing items to be introduced into catheter 10.

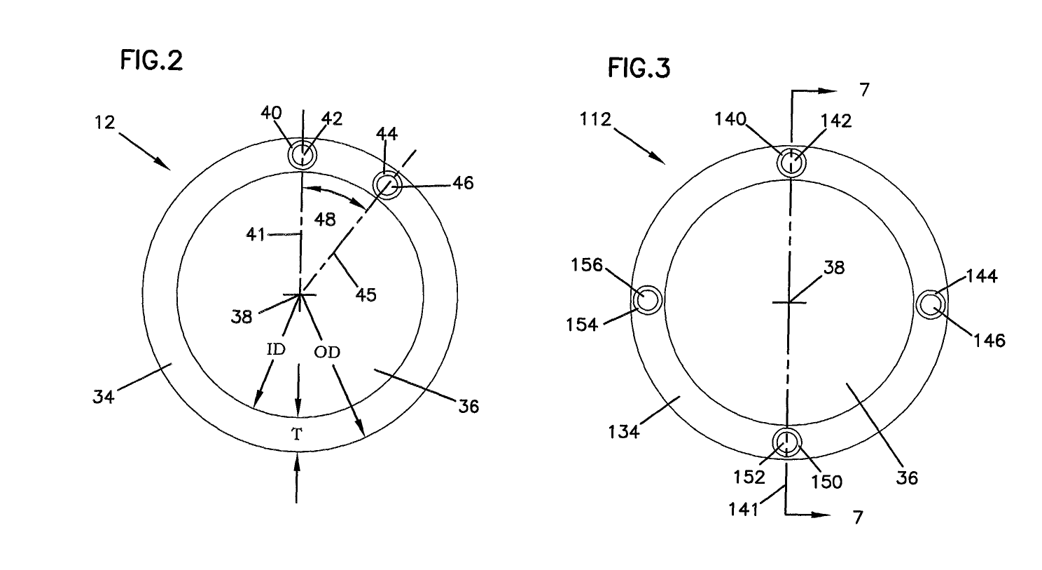

Referring now to FIG. 2, a cross-section of catheter body 12 taken at line 2-2 in FIG. 1 is shown. Catheter body 12 includes an outer wall 34 which defines a central lumen 36 having a longitudinal central axis 38. Outer wall 34 defines an inner diameter ID and an outer diameter OD separated by a wall thickness T. Inner diameter ID is preferably at least 50% as large as outer diameter OD, more preferably at least 75% as large as outer diameter OD. Within wall thickness T of outer wall 34 are a first wire 42 in a first wire lumen 40 and a second wire 46 in a second wire lumen 44. First wire lumen 40 extends at least from handle 18 to a point distal to first bending portion 24. First wire 42 is attached to a first tensioning mechanism 20 in handle 18 and extends to a point immediately distal to first bending segment 24, where it is anchored within outer wall 34. First wire 42 is sized to slide freely within first wire lumen 40 except where first wire 42 is anchored to outer wall 34. A second wire 46 within a second wire lumen 44 is located in outer wall 34, offset from first wire 42 by an angle 48. Second wire 46 is attached to a second tensioning mechanism 20 in handle 18 and extends to a point immediately distal to second bending segment 28, where it is anchored within outer wall 34. Second wire 46 is sized to slide freely within second wire lumen 44 except where second wire 46 is anchored to outer wall 34. Applying tension to first wire 42 will cause first bending portion 24 to bend in an arc along a first plane 41 defined by central axis 38 of catheter body 12 and first wire lumen 40 in first bending segment 24. Applying tension to second wire 46 will cause second bending portion 28 to bend in an arc along a second plane 45 defined by central axis 38 and second wire lumen 44 in second bending portion 28. Angle 48 defines the degree of angular separation between planes 41 and 45.

Alternatively, catheter 10 may include only first wire 42 within first wire lumen 40, with wire 42 anchored to outer wall 34 immediately distal first bending segment 24 and attached to a tensioning mechanism 20 within handle 18. By using material of lower durometer in first bending portion 24 relative to the material used in second bending segment 28, applying tension to wire 42 will initially cause first bending portion 24 to bend in an arc along plane 41. As first bending portion 24 nears its degree of maximum bend, second bending portion 28 will begin to bend in an arc along plane 41.

FIGS. 3 through 5 and 7 show several alternative embodiments of a catheter in accordance with the present invention. All of these alternative embodiments include an outer wall 34 defining a central lumen 36 having a longitudinal central axis 38. FIG. 3 shows a first alternative catheter body 112 which includes four wire lumens 142, 146, 150 and 154, with four wires 144, 148, 152 and 156, respectively, equally spaced apart about outer wall 36. Each wire 144, 148, 152 or 156 is anchored immediately distal of a bending portion. FIG. 7 is a longitudinal cross-section taken along line 7-7 in FIG. 3 and shows an example of how wires 142 and 152 might be anchored to outer wall 34 to provide bending in two directions of first bending portion 24. By having wires 142 and 152 within wire lumens 140 and 150 which both line in a plane 141 including by central axis 38, first bending portion 24 can be bent up by applying tension to wire 142, or down by applying tension to wire 152. Wires 144 and 154 are similarly opposed and attached immediately distal of second bending portion 28, allowing second bending portion to be moved toward either wire by applying tension to that wire.

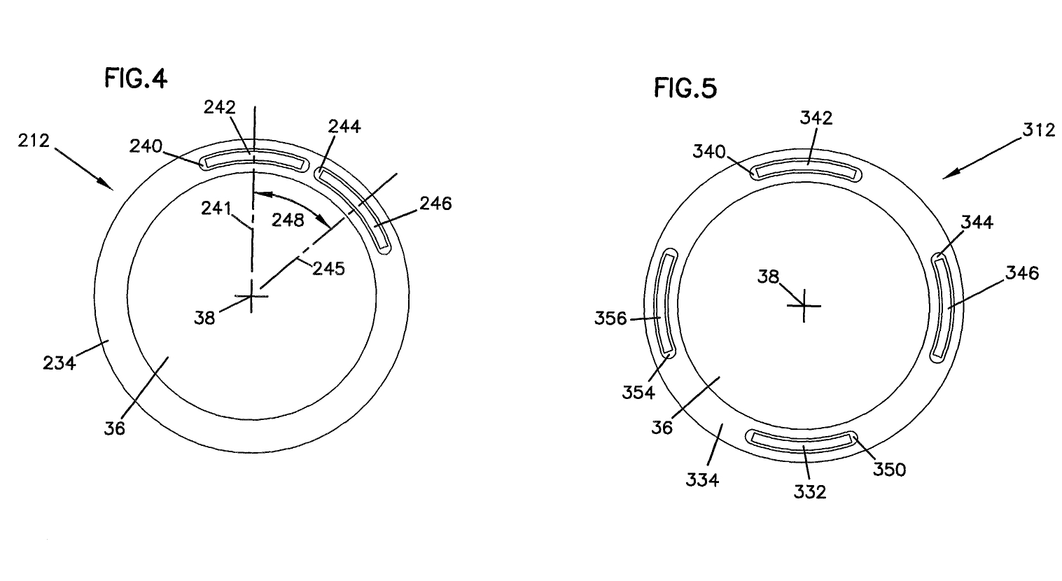

FIGS. 4 and 5 show alternative embodiments 212 and 312 of a catheter body in accordance with the present invention. Catheter bodies 212 and 312 incorporate flat tensioning members 242, 246, 342, 346, 350 and 356, in place of the round wires in the embodiments in the earlier FIGS. Catheter body 212 in FIG. 4 is similar to catheter 10 in FIG. 2, including an outer wall 234 defining an central lumen 36 with a central axis 38. A first tensioning member 242 is within a first tensioning lumen 240 and a second tensioning member 246 is within second tensioning lumen 244. Applying tension to first tensioning member 242 will cause first bending portion 24 to bend in an arc along a first plane 241 defined by central axis 38 of catheter body 212 and first tensioning lumen 240 in first bending segment 24. Applying tension to second tensioning member 246 will cause second bending portion 28 to bend in an arc along a second plane 245 defined by central axis 38 and second tensioning lumen 244 in second bending portion 28. Angle 248 defines the degree of angular separation between planes 241 and 245.

Catheter body 312 in FIG. 5 is similar to catheter body 112 in FIG. 3, with four tensioning members 342, 346, 350 and 356, within four tensioning lumen 340, 344, 348 and 354, spaced about outer wall 334.

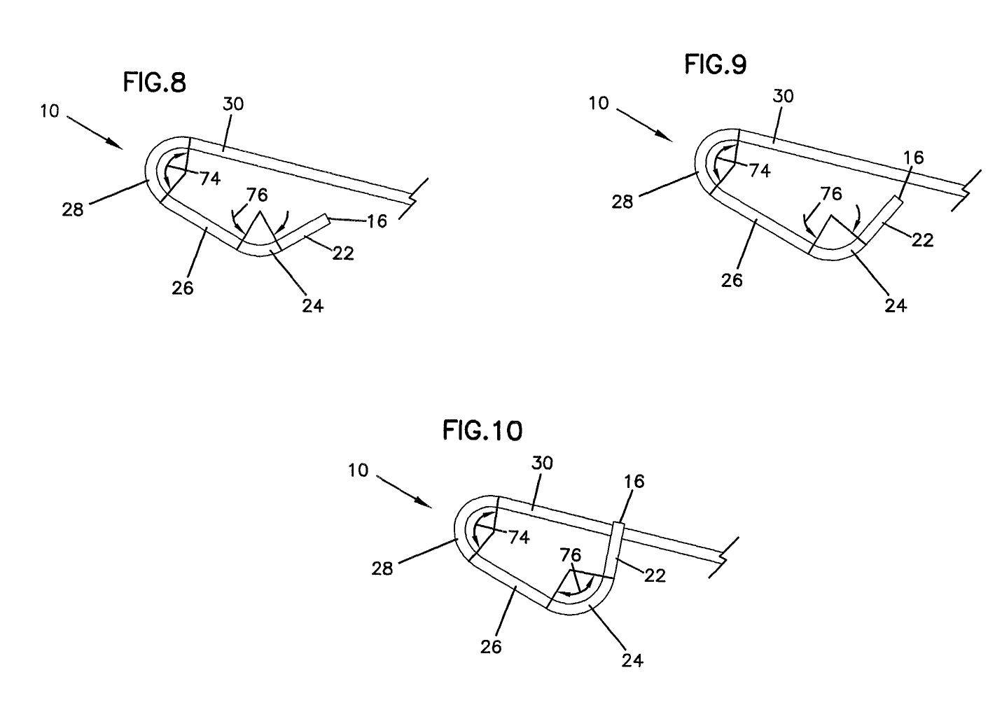

FIGS. 8 through 10 illustrate in more detail the angles through which bending segments of catheter 10 may be bent by applying tension to wires or tensioning members within outer wall 34. Second bending portion 28 is bent through an angle 74 to form a primary curve. First bending portion 24 is bent through an angle 76 to form a secondary curve. Primary curve 74 and secondary curve 76 lie in planes 45 and 41, respectively, and are thus offset from each other by angle 48. This angular offset allows distal segment 22 to overlap and cross proximal segment 30 as shown in FIG. 10.

Primary curve 74 traverses an angle in the range of one hundred forty to one hundred eighty degrees, preferably approximately one hundred and sixty degrees. Secondary curve 76 traverses an angle in the range of sixty to one hundred twenty degrees, most preferably approximately eighty degrees. As shown in FIG. 2, angle 48 is offset in a clockwise direction from plane 41 to plane 45. In other embodiments, angle 48 can be offset in a counter-clockwise direction, such that distal segment 22 in FIG. 10 would pass on the other side of proximal segment 30.

Proximal segment 30 is predominantly straight and preferably sized to extend from a femoral stick to a desired site within a patient's body. Length of segments 22 and 24 will vary depending on the site to be accessed within a patient's body. For use of catheter 10 to access sites within a patient's heart, length of mid segment 26 is preferably in the range of 1 to 9 centimeters. Similarly, for use of catheter 10 to access sites within a patient's heart, length of distal segment 22 is preferably in the range of 0.5 to 3 centimeters.

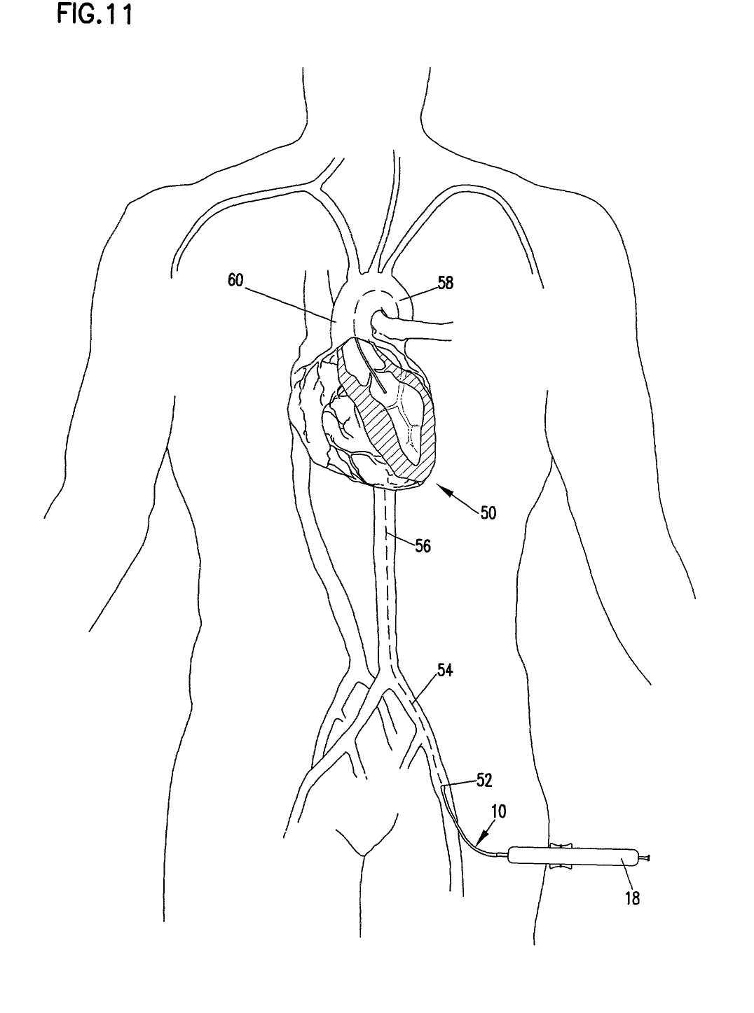

Referring now to FIGS. 11 through 16, the use of one embodiment of the catheter of the present invention to perform an endovascular catheterization of a patient to access a shunt already in place in the left ventricle of the patient's heart will be described in detail.

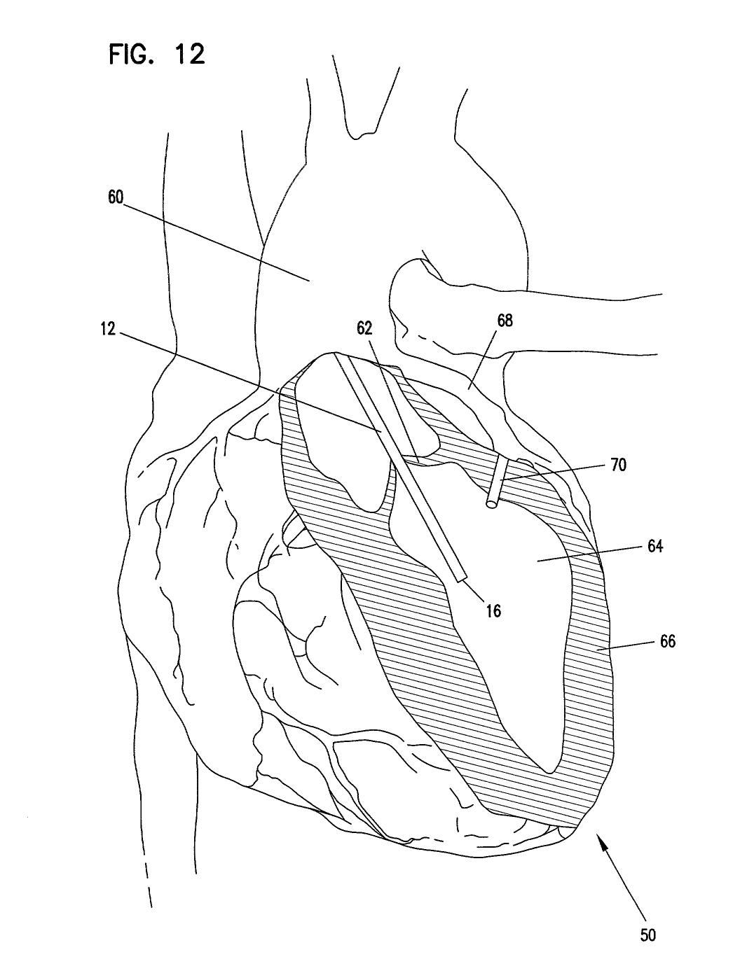

To begin such a catheterization procedure, distal end 16 of catheter 10 is inserted into the femoral artery 54 of a patient, via a site 52 in the patient's groin. Distal end 16 of is then advanced along femoral artery 54 in retrograde fashion. Upon reaching the upper most extension of the femoral artery, catheter 10 is then directed into the descending aorta 56. From descending aorta 56, catheter 10 is further advanced in retrograde fashion into the arch of aorta 58. Advancing through arch of aorta 58 retrograde, distal end 16 of catheter 10 passes through the ascending aorta 60 directly into the heart 50. Preferably, catheter 10 is advanced into a heart chamber 64 through the aortic valve 62. In FIGS. 11 and 12, the catheterization has been to the left ventricle of a patient's heart. Proximal segment 30 is preferably of sufficient length to permit the insertion of catheter 10 in femoral artery 54 of a patient and extension of catheter 10 into patient's heart 50. In FIG. 12, an enlarged view of the left ventricle of the patient is shown, with the catheter 10 entering chamber 64 from ascending aorta 60 and a shunt 70 in place in the wall 66 of heart chamber 64 being shown.

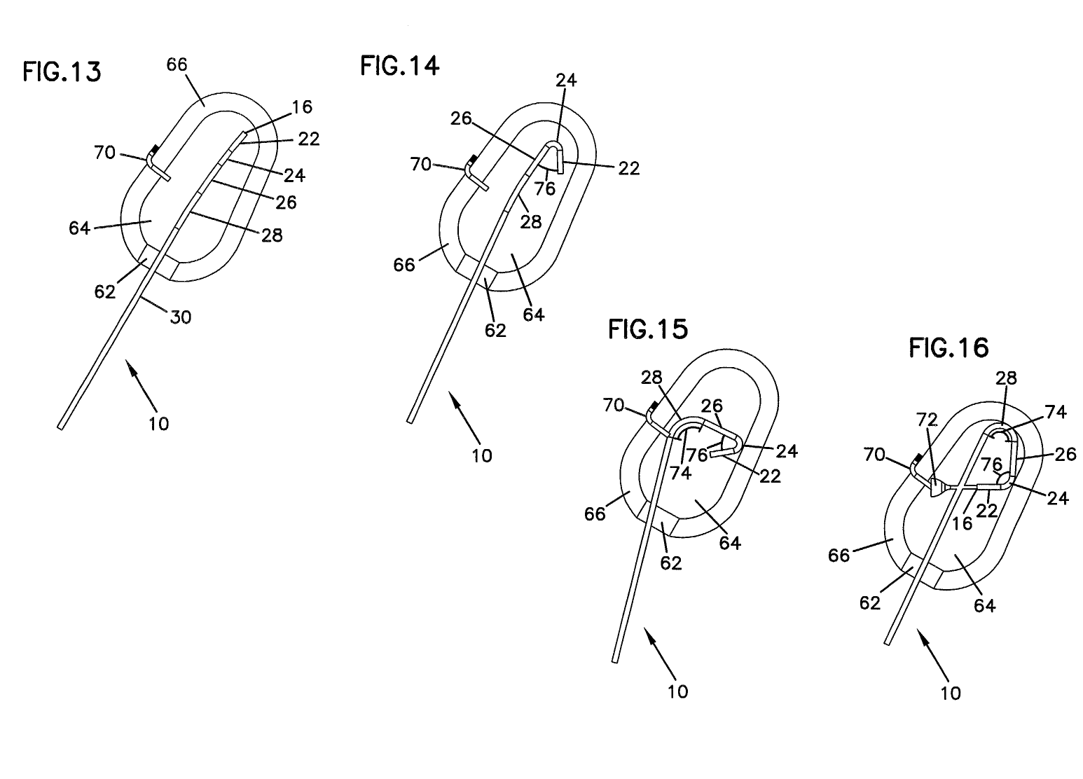

Once distal segment 22, first bending portion 24, mid segment 26, second bending portion 28 and a portion of proximal segment 30 of catheter 10 has entered heart chamber 64 via ascending aorta 60, a sequence of manipulating bending portions 24 and 28 into primary and secondary curves 74 and 76 is shown in FIGS. 13 through 16. FIG. 13 shows catheter 10 in place and unbent within heart chamber 64. Tension is applied to first wire 42 to form first bending portion 24 into secondary curve 76, as shown in FIG. 14. Tension is next applied to second wire 48 to begin forming second bending portion 28 into primary curve 74, as shown in FIG. 15. Once primary curve 74 is fully formed by bending second bending portion 28, catheter 10 is advanced further into heart chamber 64 so that second bending portion 28 is resting against wall 66, providing support to improve the stability and manipulation of catheter 10 to place distal end 16 proximate shunt 70 in heart wall 66. As shown in FIG. 16, catheter 10 has had bending portions 24 and 28 formed into secondary and primary curves 74 and 76, respectively, which are resting against heart wall 66 to position distal end 16 proximate shunt 70 and allow a snare such as basket 72 or some other type of snare, to be extended from within central lumen 36 and capture and end of shunt 70 extending into chamber 64.

Positioning of distal end 16 proximate the shunt within heart wall 66 can be accomplished by rotating catheter handle 18 and varying the degree of curvature of secondary curve 76 and primary curve 74 by varying the tension in wires 42 and 46, respectively. Once a snare such as basket 72 has been attached to shunt 70, distal end 16 can be moved directly proximate shunt 70 to allow radio-opaque contrast to be injected through shunt 70, to access coronary artery 68 at a point downflow from the shunt 70 or to perform other diagnostic procedures.

It is anticipated that catheter 10 can be used to access other locations within a patient's body and that the length of proximal segment 30, mid segment 26 and distal segment 22 may be varied to suit the location desired to be accessed. Additionally, amount of bend available to form primary curve 74 and secondary curve 76 in bending portions 28 and 24 may be varied to suit the location to be accessed. Further, angle 48 defining the angular separation between first plane 41 and second plane 45 may be varied to suit accessing a different location. While its preferred use is for coronary diagnostic procedures, the present invention can be used for other medical procedures where precise control of a catheter tip is desired. It will be appreciated that the size, shape and bend locations can be varied to correspond to different applications.

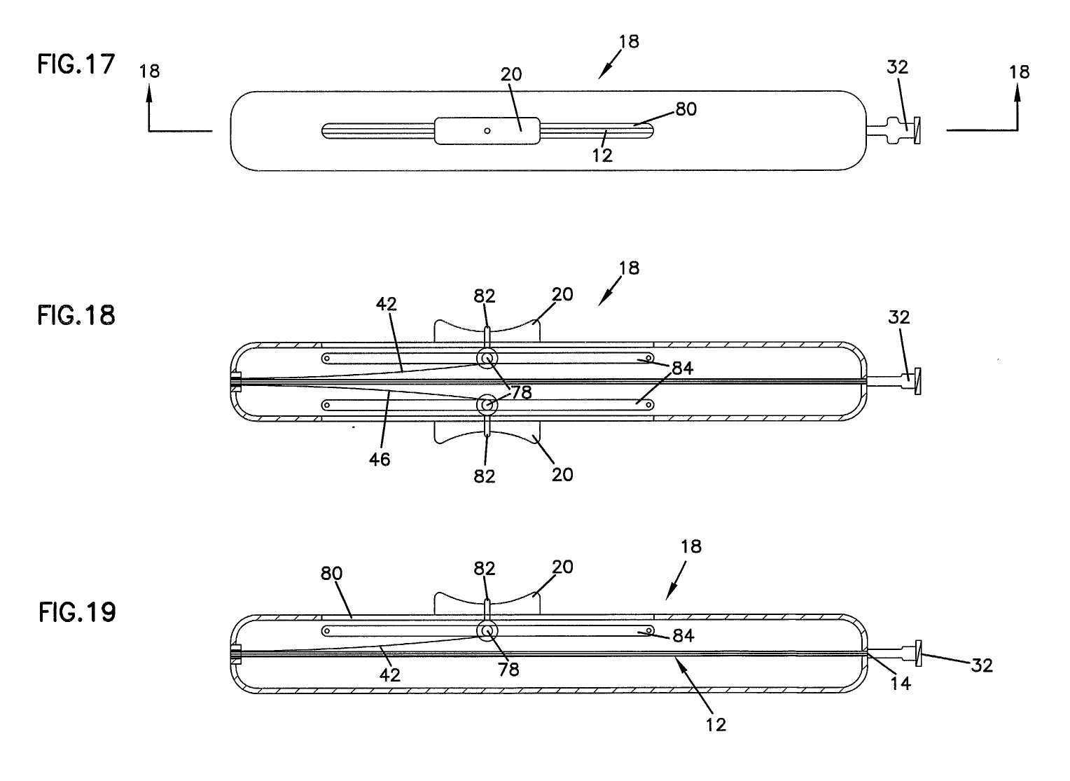

Referring now to FIGS. 17 and 18, handle 18 is shown in detail for use with catheter 10, permitting tension to be applied to wires or tensioning members within outer wall 34. Handle 18 is shown with two slides 20, each of which may be moved along an opening 80 and which are attached to first and second wires 42 and 46 by anchors 82. Moving a slide 20 proximally along opening 80 applies tension to the wire attached to that slide, while moving the same slide 20 distally will release tension in the attached wire. Anchors 82 include an extension 78 which rides along an internal guide 84. As shown in FIGS. 17 and 18, catheter body 12 extends through handle 18, with proximal end 14 at the rear of handle 18 where a port 32 is mounted. Port 32 allows access to the central lumen 36 for inserting tools, diagnostic devices, fluids or other similar objects into catheter 10 for insertion within the patient's body at the site being accessed with catheter 10. While slides 20 are shown on opposite sides of handle 18, slides 20 may be attached to wires 42 and 46 which have an angular offset of less than one hundred eighty degrees. Handle 18 may be used with any single or dual wire embodiment of a catheter in accordance with the present invention. A locking mechanism, such as a thumb screw or other releasable device, not shown in the FIGS., may be used to hold slide 20 so that the desired amount of tension is being applied to wire 42 or 46.

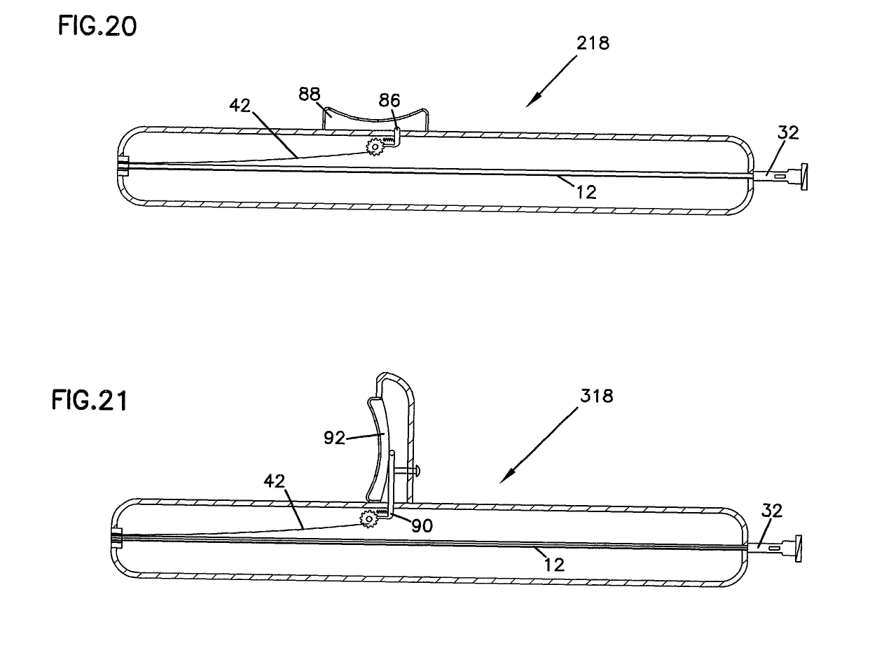

Referring now to FIGS. 19 through 23, FIG. 19 shows an alternative handle 118, which provides a single slide 20 for applying tension to a wire 42. FIG. 20 shows another alternative handle 218, similar to 118 in that only a single actuator 88 is provided to apply tension to wire 42. Actuator 88 actuates a ratcheting mechanism 86 to apply tension to wire 42 and hold a desired amount of tension. When the procedure being performed with catheter 10 is complete, the ratchet releases the tension on the wire, allowing the bending segments of the catheter to return to their original shapes. Sliding actuator 88 proximally and releasing it causes ratchet 86 to increase and hold tension in wire 42. Sliding actuator 88 proximally and holding actuator 88 in this position releases the tension in wire 42.

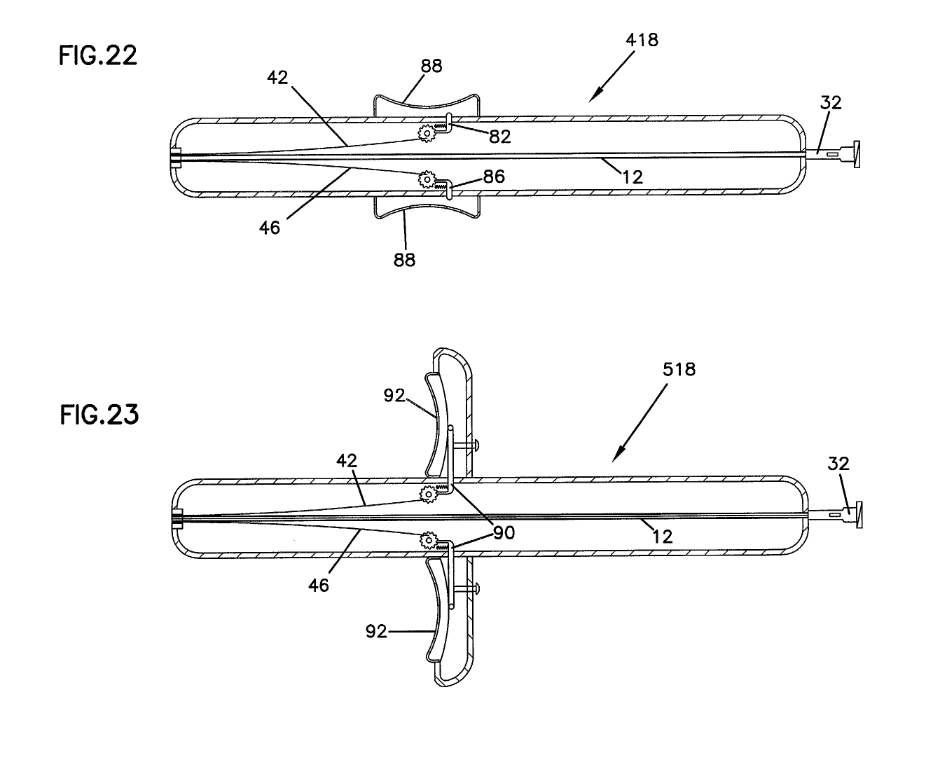

FIG. 21 also shows an alternative handle 318, which is adapted to provide tension to a single wire 42 of catheter 10. Handle 318 includes a trigger-style actuator 92 to apply tension to wire 42. Actuator 92 actuates a ratcheting mechanism 90 to apply, hold and release tension on wire 42, in a similar fashion as that described above with regard to actuator 88 and ratchet 86. FIG. 22 shows a further alternative handle 418, which incorporates two actuators 88 actuating two ratcheting mechanisms 86 to apply, hold and release tension on wires 42 and 46. FIG. 23 shows a further embodiment handle 518, which incorporates two trigger-style actuators 92 actuating two ratcheting mechanisms 90 to apply, hold and release tension on wires 42 and 46.

Additional embodiments of handle 18 are anticipated which will incorporate actuators and tensioning mechanisms for each of the wires or tensioning devices which may be within outer wall 34 of a catheter 10.

Having described preferred aspects and embodiments of the present invention, modifications and equivalents of the disclosed concepts may readily occur to one skilled in the art. However, it is intended that such modifications and equivalents be included within the scope of the claims which follow.

What is claimed is:

1. A catheter comprising:
a body extending between a distal end and a proximal end;
the body including a central lumen defined by an outer wall, the central lumen extending longitudinally from the proximal end to the distal end and permitting fluid communication between the ends, the outer wall made of a resilient, flexible material and including a bend portion adjacent the distal end where the outer wall has greater flexibility;
the body further including a tensioning member lumen extending longitudinally through the outer wall;
a tensioning member within the tensioning member lumen extending from the proximal end toward the distal end to a point distal to the bend portion of the body and anchored to the outer wall within the tensioning member lumen at the point;
wherein applying tension to the tensioning member deflects the distal end along a plane defined by the tensioning member and a central axis of the body, in the direction of the tensioning member, the bend portion forming a bend along the plane.

2. The catheter of claim 1, wherein the bending portion is a first bending portion and the catheter body also includes a second bending portion located between the proximal end and the first bending portion, the bend formed by the first bending portion is a first bend and tensioning the tensioning member further causes the seconding bending portion to form a second bend along the plane and deflect the distal end of the catheter.

3. The catheter of claim 2, wherein the first bending portion is made of a material with a lower durometer than the second bending portion so that tensioning the tensioning member causes the first bending portion to bend before the second bending portion.

4. The catheter of claim 3, wherein tensioning the tensioning member bends the first bend to a maximum arc of approximately one hundred eighty degrees.

5. The catheter of claim 3, wherein tensioning the tensioning member bends the first bending to a maximum arc of approximately one hundred sixty degrees.

6. The catheter of claim 3, wherein tensioning the tensioning member bends the second bend to a maximum arc of approximately one hundred degrees.

7. The catheter of claim 3, wherein tensioning the tensioning member bends the second bend to a maximum arc of approximately eighty degrees.

8. The catheter of claim 1, wherein a handle is mounted to body at the proximal end, the handle allowing fluid communication with the central lumen and providing a tensioning mechanism for tensioning the tensioning member.

9. The catheter of claim 8 wherein the tensioning mechanism further includes a releasable lock for maintaining a desired amount of tension in the tensioning member.

10. The catheter of claim 1, wherein the bending portion is a first bending portion and a second bending portion is located between the first bending portion and the proximal end, the tensioning member lumen is a first tensioning member lumen and a second tensioning member lumen extends parallel to the first tensioning member lumen angularly displaced about the outer wall from the first tensioning member lumen, the tensioning member is a first tensioning member and a second tensioning member is within the second tensioning member lumen and anchored to the outer wall within the second tensioning member lumen at a point distal to the second bend portion, so that a tension applied to the second tensioning member deflects the distal end along a second plane defined by the second tensioning member and the central axis of the body in the direction of the second tensioning member, the second bend portion forming an bend along the second plane.

11. The catheter of claim 10, wherein tensioning the first tensioning member bends the first bend to a maximum arc of approximately one hundred eighty degrees.

12. The catheter of claim 10, wherein tensioning the first tensioning member bends the first bend to a maximum arc of approximately one hundred sixty degrees

13. The catheter of claim 10, wherein tensioning the second tensioning member bends the second bend to a maximum arc of approximately one hundred degrees, preferably approximately eighty degrees.

14. The catheter of claim 10, wherein tensioning the second tensioning member bends the second bend to a maximum arc of approximately eighty degrees.

15. The catheter of claim 10, wherein a handle is mounted to the body at the proximal end, the handle allowing fluid communication with the central lumen and providing a tensioning mechanism for tensioning the first and second tensioning members independently.

16. The catheter of claim 15, wherein the handle further includes a releasable lock for independently maintaining a desired amount of tension in the first tensioning member and in the second tensioning member.

17. The catheter of claim 10, wherein a distal segment is defined between the distal end and the first bending portion, a mid segment is defined between the first and second bending portions and a proximal segment is defined between the second bending portion and the proximal end, the distal segment having a length between 0.5 centimeters and 3 centimeters.

18. The catheter of claim 17, wherein the mid segment has a length between 1 centimeter and 9 centimeters.

19. The catheter of claim 10, wherein the angular displacement between the first and second tensioning members is between approximately ten degrees and approximately fifty degrees. approximately thirty degrees.

20. The catheter of claim 10, wherein the angular displacement between the first and second tensioning members is approximately thirty degrees.

21. A method of accessing a location on a heart wall comprising the steps of:
providing a catheter including a body with a central lumen defined by an outer wall, a distal end, a proximal end, a first bending portion and a second bending portion;
advancing the distal end, the first bending portion and the second bending portion into a chamber of a heart including the heart wall with the location to be accessed;
tensioning a first tensioning member within a first tensioning member lumen in the outer wall, causing the first bending portion to bend in an arc and deflect the distal end of the catheter; and
tensioning a second tensioning member within a second tensioning member lumen in the outer wall, causing the second bending portion to bend in an arc and deflect the distal end of the catheter to align the distal end with the location.

22. A method of accessing a location on a heart wall comprising the steps of:
providing a catheter including a body with a central lumen defined by an outer wall, a distal end, a proximal end, a first bending portion and a second bending portion, the first bending portion made from a lower durometer material than the second bending portion;
advancing the distal end, the first bending portion and the second bending portion into a chamber of a heart including the heart wall with the location to be accessed;
tensioning a tensioning member within a tensioning member lumen in the outer wall, causing the first bending portion to bend in an arc and deflect the distal end of the catheter; and
further tensioning the tensioning member causing the second bending portion to bend in an arc and deflect the distal end of the catheter to align the distal end with the location.
